(19) United States
(12) Patent Application Publication
(10) Pub. No.: US 2013/0244305 A1
(43) Pub. Date: Sep. 19, 2013

(54) SOLVENT AND METHOD FOR CO2 CAPTURE FROM FLUE GAS

(71) Applicants: ALSTOM TECHNOLOGY LTD., Baden (CH); CO2 SOLUTIONS INC., Quebec City (CA)

(72) Inventors: Geert F. VERSTEEG, Enschede PD (NL); Sylvie FRADETTE, Pintendre (CA); Olivera CEPERKOVIC, Quebec (CA); Frederic VITSE, Knoxville, TN (US); Stephen A. BEDELL, Knoxville, TN (US); Barath BABURAO, Knoxville, TN (US)

(21) Appl. No.: 13/868,714

(22) Filed: Apr. 23, 2013

Related U.S. Application Data
(63) Continuation of application No. 13/195,056, filed on Aug. 1, 2011.
(60) Provisional application No. 61/383,046, filed on Sep. 15, 2010.
(60) Provisional application No. 61/637,595, filed on Apr. 24, 2012.
(60) Provisional application No. 61/782,250, filed on Mar. 14, 2013.

(51) Int. Cl. B01D 53/84; C12N 9/88; C09K 3/00

(52) U.S. Cl. CPC B01D 53/84; C09K 3/00; C12N 9/88
USPC 435/232; 252/184; 423/228; 435/266

(57) ABSTRACT

The present disclosure describes the efficient use of a catalyst, an enzyme for example, to provide suitable real cyclic capacity to a solvent otherwise limited by its ability to absorb and maintain a high concentration of CO₂ captured from flue gas. This invention can apply to non-promoted as well as promoted solvents and to solvents with a broad range of enthalpy of reaction.

### FIELD OF THE INVENTION

This application is a continuation of U.S. patent application Ser. No. 13/195,056, filed Aug. 1, 2011, which claims priority of U.S. provisional application 61/383,046, filed Sep. 15, 2012, also claiming priority of U.S. provisional application 61/637,595 filed Apr. 24, 2012, and U.S. provisional 61/782,250, filed Mar. 14, 2013, all of which are incorporated herein by reference in their entirety.

### BACKGROUND OF THE INVENTION

The present disclosure relates to the use of catalytically enhanced solvents for CO₂ capture from flue gas, thus avoiding the needs for promoters or higher enthalpy of reaction solvents.

For flue gas applications, the process conditions (dilute CO₂ concentrations, low partial pressures, low heat capacity of the flue gas) are such that the absorption process is limited either by low absorption rates or by excessive increase of the temperature in the absorber during the corresponding exothermic reactions.

In the past, these two issues have been addressed by the use of solvents with higher enthalpy of absorption. The higher enthalpy of absorption is generally associated with the stronger alkaline properties of the solvent (higher pKa) and therefore, increased rate of reaction as well as higher solubility of CO₂ in the solvent. In particular, some prominent work in CO₂ capture from flue gas with amine-based solvent recommends higher enthalpy of reaction solvents for flue gas application [Rochelle].

Unfortunately, higher enthalpy of reaction solvents have a drawback, in they participate to the increase in the energy demand for regeneration of the solvent. The improved affinity of the CO₂ solvent in the absorber becomes a disadvantage when it comes to reverse the reaction in the regenerator. Therefore, there is a trade-off with which to deal.

### SUMMARY OF THE INVENTION

The present invention involves the efficient use of a catalyst, an enzyme for example, to reduce the constraints associated with the trade-off described above, thus providing suitable real cyclic capacity to a solvent otherwise limited by its ability to absorb and maintain a high concentration of CO₂ captured from flue gas. This invention can apply to non-promoted as well as promoted solvents and to solvents with a broad range of enthalpy of reaction.

### BRIEF DESCRIPTION OF THE DRAWINGS

FIG. 1 is a schematic depiction of a conventional system for removal of CO₂ from a gas stream.

FIG. 2 is a plot of theoretical cyclic capacities (based on thermodynamic CO₂ loading capacities) as a function of the acid dissociation constant (pKa) of different amines.

### DESCRIPTION OF PREFERRED EMBODIMENTS

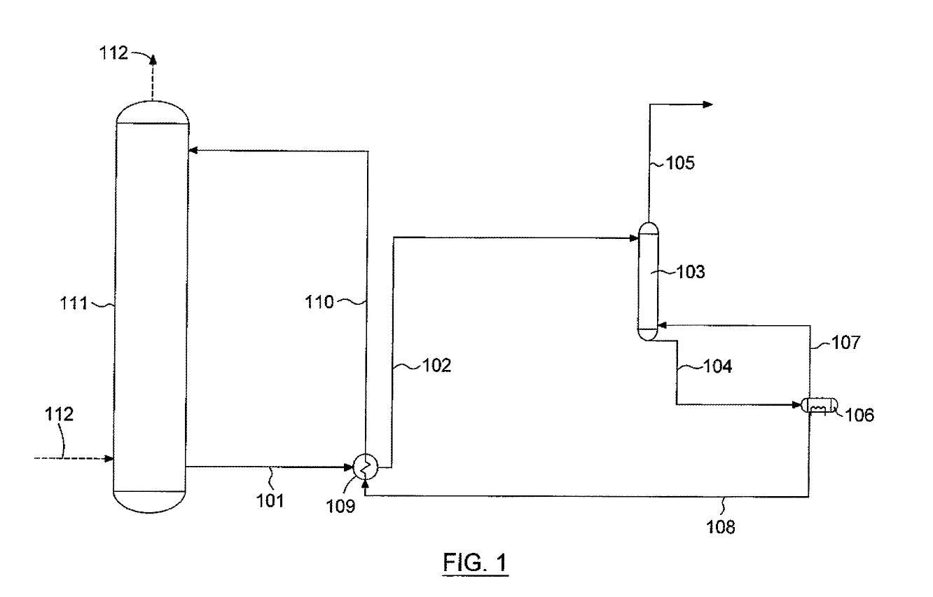

FIG. 1 illustrates a conventional system for removal of CO₂ from a gas stream. The system comprises an absorber column (absorber) 111, wherein a gas stream (e.g., a flue gas stream) 112 containing CO₂ is contacted, for example in a countercurrent mode, with a solvent solution 110, such as an amine-based solvent. In the absorber, CO₂ from the gas stream is absorbed in the solvent. Used solvent enriched in CO₂ leaves the absorber via line 101. The CO₂-enriched solvent is passed via a heat exchanger 109 and line 102 to a regenerator 103, wherein the used solvent is stripped of CO₂ by breaking the chemical bond between the CO₂ and the solution. Regenerated solvent leaves the regenerator bottom via line 104. Removed CO₂ and water vapor leaves the process at the top of the regenerator via line 105. In addition, a condenser may be arranged at the top of the regenerator to prevent water vapor from leaving the process.

Regenerated solvent is passed to a reboiler 106 via line 104. In the reboiler, located at the bottom of the regenerator, the regenerated solvent is boiled to generate vapor 107, which is returned to the regenerator to drive the separation of CO₂ from solvent. In addition, reboiling may provide for further CO₂ removal from the regenerated solvent.

Following reboiling, the reboiled and thus heated solvent is passed via line 108 to a heat exchanger 109 for heat-exchanging with the used solvent from the absorber. Heat exchanging allows for heat transfer between the solutions, resulting in a cooled reboiled solvent and a heated used solvent. The reboiled and heat-exchanged solvent is thereafter passed to the next round of absorption in the absorber. Before being fed to the absorber, the solvent 110 may be cooled to a temperature suitable for absorption. Accordingly, a cooler may be arranged near the absorber solvent inlet (not shown).

Examples of conventional amine-based solvents include, for example, amine compounds such as monoethanolamine (MEA), diethanolamine (DEA), methyldiethanolamine (MDEA), diisopropylamine (DIPA) and aminoethoxyethanol (diglycolamine) (DGA). The most commonly used amines compounds in industrial plants are the alkanolamines MEA, DEA, MDEA and some blends of conventional amines with promoters (e.g., piperazine) and/or inhibitors.

A typical amine-based solvent for flue gas applications absorbs CO₂ at temperatures around 100-140° F. Below this lower temperature, the kinetics of absorption are limited or slower, above this upper temperature, the solubility of CO₂ in the solvent is rapidly diminished. The temperature of the solvent inside the absorber can be higher than its inlet or outlet temperatures due the exothermic nature of the reaction of absorption. This can lead to an internal thermodynamic pinch and poor utilization of the absorber column for mass transfer.

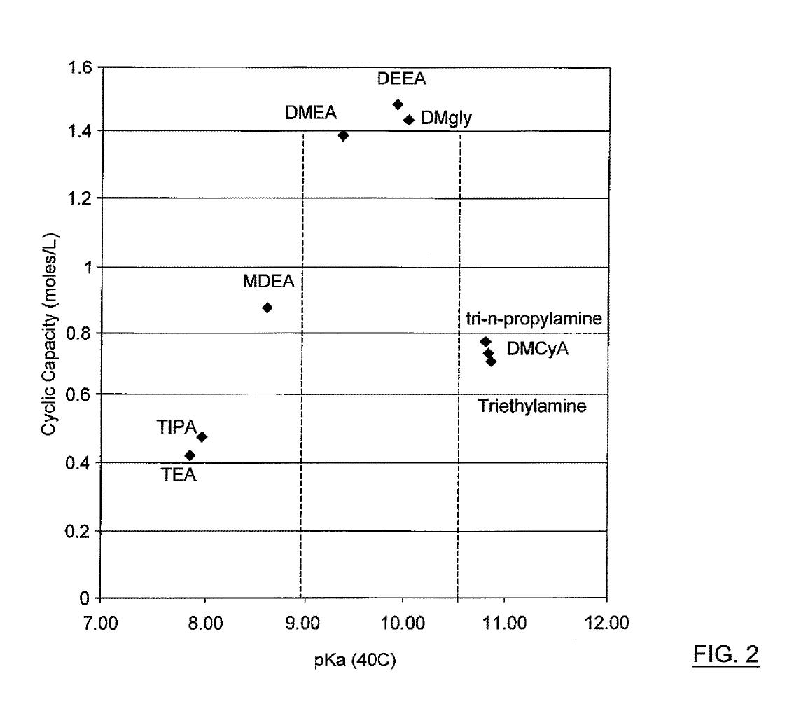

This invention targets solvents with relatively high theoretical cyclic capacities (based on thermodynamic CO₂ loading capacities), for example cyclic capacities greater than about 1 mole/liter, but with limited ability to absorb CO₂ under real process conditions (slow absorption rate and/or temperature-altered solubility due to exothermic reaction in the absorber), therefore not achieving a significant percentage of the theoretical cyclic capacity. For example, FIG. 2 is a plot of theoretical cyclic capacity as a function of the acid dissociation constant (pKa) of different amines. As shown in FIG. 2, other tertiary amines such as, for example, DMEA (dimethylethanolamine), DEEA (diethylethanolamine), and DMgly (dimethylglycine), can have higher cyclic capacities than MDEA. We have observed that these amines typically have a pKa (40° C.) in the range of about 9 to about 10.5. The amines at the top of the curve have greater capacity than MDEA, but have previously been thought to be too slow to react in a reasonably sized absorber.

By using a catalyst that enhances the kinetics of CO₂ absorption at lower temperatures, the process conditions in the absorber can be optimized to increase the real cyclic capacity of the solvent to a higher percentage of the theoretical cyclic capacity (as defined by thermodynamics). Such catalysts may include, for example biocatalysts such as carbonic anhydrase or its analogs. There is no limitation to how low the temperature should be, at which the catalyst should enhance the kinetics, however, from a practical perspective, the following temperature range can be recommended. The catalyst should allow achieving increased CO₂ loadings compared to a non-catalyzed solvent at temperatures in the range of 80-140° F. In particular, for any solvent, a catalyst that allows reaching the same or higher absorption rate but at lower temperature is beneficial.

With a catalytically-enhanced solvent, optimization of the process for higher cyclic capacities can be achieved by:
Lowering the inlet temperature of the solvent entering the absorber. The entire column is therefore cooler, thus increasing the solubility of CO₂ but without penalizing the absorption rate. This leads to increased real rich loading for a fixed lean loading compared to a non-catalyzed solvent.
Lowering the temperature of the solvent within the absorber by using intercooling (e.g., cooling coils or other heat exchanger within the absorber tower) or/and intercooling-recycling (e.g., withdrawal of a portion of the solvent from the absorber tower, cooling the portion, and re-injecting it back into the absorber column). Part of the column is therefore cooler, thus increasing the solubility of CO₂ but without penalizing the absorption rate. This leads to increased real rich loading for a fixed lean loading compared to a non-catalyzed solvent.
Lowering the liquid-to gas flow rate ratio. This can promote lower temperature in the bottom of the absorber column by allowing the temperature bulge associated with the exothermic reaction to be at the top of the absorber. Part of the column is therefore cooler, thus increasing the solubility of CO₂ but without penalizing the absorption rate. This leads to increased real rich loading for a fixed lean loading compared to a non-catalyzed solvent.

### EXAMPLES

In this example a catalytically enhanced MDEA is selected and compared it to MDEA-Pz, where Pz plays the role of a promoter. This is for illustration only, the invention can apply to MDEA, MDEA-Pz, and, in general, to any solvent that show high enough theoretical cyclic capacity for a specified degree of CO₂ separation from flue gas.

Below the theoretical cyclic capacity of MDEA and MDEA-Pz are compared at a specific process temperature and flue gas composition:
PCO₂ inlet flue gas of 15 kPa
   the solvent theoretical cyclic capacity of MDEA is:
0.38 at 95° F.
0.32 at 105° F.
0.27 at 115° F.
0.22 at 125° F.
   the solvent theoretical cyclic capacity of MDEA-Pz is:
0.47 at 95° F.
0.44 at 105° F.
0.39 at 115° F.
0.36 at 125° F.
   For this application, it is proposed to remove 90% from a flue gas. The selected liquid to gas ratio is 3.36 kg/hr/kg/hr for a minimum real cyclic capacity of ≈0.30 mol CO₂/mol amine for MDEA-Pz and ≈0.32 mol CO₂/mol amine for MDEA.

Therefore, at all temperatures (95-125° F.), MDEA-Pz can theoretically accomplish the separation, while MDEA can only achieve the separation at 95° F. The liquid to gas ratio for MDEA solvent can be increased to achieve the capture rate with a cyclic capacity of less than 0.32 mol/mol but this entails a higher liquid to gas ratio and a corresponding increased energy penalty. The corresponding energy penalties are reported in Table 1 and Table 2.

TABLE 1
Reboiler duty associated with 90% CO₂ capture with
MDEA-Pz from a flue gas containing 15 kPa CO₂

| | Rich | Rich | Lean, | Reboiler | Regen |
|---|---|---|---|---|---|
| L/G, | outlet | loading, | loading, | duty | overhead |
| lb/lb | T, deg F. | mol/mol | mol/mol | Gj/Tonne | T, deg F. |
| 3.4 | 95 | 0.47 | 0.17 | 2.31 | 194.4 |
| 3.4 | 105 | 0.435 | 0.13 | 2.52 | 199.4 |
| 3.4 | 115 | 0.39 | 0.08 | 2.81 | 204.7 |
| 3.4 | 125 | 0.36 | 0.05 | 3.00 | 207.7 |

TABLE 2
Reboiler duty associated with 90% CO₂ capture
with MDEA from a flue gas containing 15 kPa CO₂

| | Rich | Rich | Lean | Reboiler | Regen |
|---|---|---|---|---|---|
| L/G, | outlet | loading, | loading, | duty | overhead |
| lb/lb | T, deg F. | mol/mol | mol/mol | Gj/Tonne | T, deg F. |
| 3.41 | 95 | 0.38 | 0.05 | 1.98 | 190.0 |
| 3.53 | 105 | 0.33 | 0.01 | 2.30 | 203.3 |
| 4.15 | 115 | 0.27 | 0.00 | 2.57 | 209.1 |
| 5.24 | 125 | 0.22 | 0.01 | 2.88 | 213.4 |

From these two Tables, it is seen that a catalyst providing to MDEA a cyclic capacity equivalent to the theoretical cyclic capacity allows for a reduced energy penalty as compared to a promoted solvent with a higher enthalpy of reaction. In this specific case, catalyzed MDEA is expected to have an enthalpy of reaction of 42 kJ/mol CO₂ versus ≈70-80 kJ/mol CO₂ for MDEA-Pz solvent. One can also notice that a catalyst that enhances the kinetics enough to reach the theoretical cyclic capacity at low temperatures (95° F. in this case) offers improved energy numbers at the same solvent circulation rate (liquid to gas ratio) as the promoted solvent. However, if the temperature at which the catalyst performs is increased, the separation can only be achieved at the cost of a higher liquid to gas ratio and a corresponding reduction in the energy saving as compared to a promoted catalyst (in this case 15% reduction in energy demand at 95° F. versus only 6% reduction in energy demand at 125° F.).

In a real application, it is not expected that the theoretical cyclic capacity can be reached. Due to volume and contact time limitation, the real cyclic capacity will only be a percentage of the theoretical cyclic capacity. In Table 3 and 4, it is demonstrated how a catalyst, by impacting the achievable approach to the thermodynamic equilibrium loading at the absorber bottom column, can improve the energy performance of the solvent. The process conditions remain identical as the one listed earlier.

TABLE 3
Energy demand of MDEA-Pz as a function of the achievable CO₂
loading at the absorber outlet

| | Rich | Rich | Lean | Reboiler | Regen | |
|---|---|---|---|---|---|---|
| L/G, | outlet T, | loading, | loading, | duty | overhead T, | % |
| lb/lb | deg F. | mol/mol | mol/mol | Gj/Tonne | deg F. | ATE* |
| 3.36 | 95 | 0.47 | 0.17 | 2.31 | 194.4 | 100 |
| 3.36 | 95 | 0.42 | 0.12 | 2.60 | 200.7 | 90 |
| 3.36 | 95 | 0.38 | 0.07 | 2.93 | 205.3 | 80 |
| 3.36 | 95 | 0.33 | 0.02 | 3.29 | 208.0 | 70 |

*Approach to equlibrium

TABLE 4
Enegry demand of catalyzed MDEA as a function of the
achievable CO₂ loading at the absorber outlet

| | Rich | Rich | Lean | Reboiler | Regen | |
|---|---|---|---|---|---|---|
| L/G, | outlet T, | loading, | loading, | duty | overhead T, | % |
| lb/lb | deg F. | mol/mol | mol/mol | Gj/Tonne | deg F. | ATE* |
| 3.41 | 95 | 0.38 | 0.05 | 1.98 | 190.0 | 100 |
| 3.41 | 95 | 0.34 | 0.01 | 2.23 | 201.8 | 90 |
| 3.70 | 95 | 0.27 | 0.01 | 2.40 | 206.3 | 80 |
| 4.34 | 95 | 0.27 | 0.01 | 2.58 | 209.7 | 70 |

*Approach to equlibrium

For a representative approach to equilibrium of 70-80%, the reduction in energy demand at 95° F. is between 18 and 21% when using the catalytically enhanced MDEA as compared with the Pz promoted MDEA.

At a higher temperature than 95° F. (not shown here), the same trends are expected, however, the benefit in energy reduction is expected to be less due to the need for a higher solvent circulation rate associated with the lower cyclic capacity of the solvent.

In the above example, it is demonstrated that a catalytically enhanced solvent such as MDEA can perform better than a chemically promoted solvent (such as MDEA-Pz). An energy penalty reduction of 20% or above is achievable if the catalytic enhancement occurs at low enough temperature. At a higher temperature, the benefit is also seen but with an expected energy reduction as the solvent circulation rate needs to be increased to achieve a specified degree of CO₂ separation (e.g. 90%). This invention can apply to any amine-based solvent, promoted. This invention is most suitable to solvents with a lower enthalpy of reaction.

While the invention has been described with reference to various exemplary embodiments, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims.

1. A solvent solution for the capture of CO₂ from a flue gas stream, the solvent solution including:
an amine solvent; and
a catalyst achieving increased CO₂ loadings in the amine solvent as compared to a non-catalyzed solvent at temperatures in the range of 80-140° F.

2. The solvent solution of claim 1, wherein the catalyst is a biocatalyst.

3. The solvent solution of claim 1, wherein the biocatalyst is carbonic anhydrase or an analog thereof.

4. The solvent solution of claim 1, wherein the amine solvent has a theoretical cyclic capacity greater than or equal to about 1 mole/liter.

5. The solvent solution of claim 1, wherein the amine solvent has an acid dissociation constant (pKa) greater than or equal to about 9 and less than or equal to about 10.5.

6. The solvent solution of claim 1, wherein the amine solvent is selected from the group including DMEA (dimethylethanolamine), DEEA (diethylethanolamine), and DMgly (dimethylglycine).

7. A method of reducing energy demand of a system for capturing CO₂ from a flue gas stream using an amine solvent, the method comprising:
applying a CO₂ lean solvent solution to a CO₂ rich flue gas stream in an absorber column to provide a CO₂ rich solvent solution and a CO₂ lean flue gas stream, the solvent solution including:
an amine solvent, and
a catalyst achieving increased CO₂ loadings in the amine solvent as compared to a non-catalyzed solvent at temperatures in the range of 80-140° F.; and
reducing a temperature of the CO₂ lean solvent solution provided to the absorber column, thereby increasing the solubility of CO₂ within the absorber column.

8. The solvent solution of claim 7, wherein the catalyst is a biocatalyst.

9. The solvent solution of claim 7, wherein the biocatalyst is carbonic anhydrase or an analog thereof.

10. The solvent solution of claim 7, wherein the amine solvent has a theoretical cyclic capacity greater than or equal to about 1 mole/liter.

11. The solvent solution of claim 7, wherein the amine solvent has an acid dissociation constant (pKa) greater than or equal to about 9 and less than or equal to about 10.5.

12. The solvent solution of claim 7, wherein the amine solvent is selected from the group including DMEA (dimethylethanolamine), DEEA (diethylethanolamine), and DMgly (dimethylglycine).

13. A method of reducing energy demand of a system for capturing CO₂ from a flue gas stream using an amine solvent, the method comprising:
applying a CO₂ lean solvent solution to a CO₂ rich flue gas stream in an absorber column to provide a CO₂ rich solvent solution and a CO₂ lean flue gas stream, the solvent solution including:
an amine solvent, and
a catalyst achieving increased CO₂ loadings in the amine solvent as compared to a non-catalyzed solvent at temperatures in the range of 80-140° F.; and
lowering the temperature of the solvent solution within the absorber column, thereby increasing the solubility of CO₂ within the absorber column.

14. The method of claim 13, wherein the solvent temperature is lowered using at least one of recycling and intercooling of the solvent solution and recycling of the solvent solution.

15. The solvent solution of claim 13, wherein the catalyst is a biocatalyst.

16. The solvent solution of claim 13, wherein the biocatalyst is carbonic anhydrase or an analog thereof.

17. The solvent solution of claim 13, wherein the amine solvent has a theoretical cyclic capacity greater than or equal to about 1 mole/liter.

18. The solvent solution of claim 13, wherein the amine solvent has an acid dissociation constant (pKa) greater than or equal to about 9 and less than or equal to about 10.5.

19. The solvent solution of claim 13, wherein the amine solvent is selected from the group including DMEA (dimethylethanolamine), DEEA (diethylethanolamine), and DMgly (dimethylglycine).

20. A method of reducing energy demand of a system for capturing CO₂ from a flue gas stream using an amine solvent, the method comprising:
applying a CO₂ lean solvent solution to a CO₂ rich flue gas stream in an absorber column to provide a CO₂ rich solvent solution and a CO₂ lean flue gas stream, the solvent solution including:
an amine solvent, and
a catalyst achieving increased CO₂ loadings in the amine solvent as compared to a non-catalyzed solvent at temperatures in the range of 80-140° F.; and
lowering the flow rate ration of the CO₂ lean solvent and the CO₂ rich flue gas stream within the absorber to promote a lower temperature at a bottom region of the absorber column by allowing a temperature bulge associated with an exothermic reaction between the CO₂ lean solvent and the CO₂ rich flue gas stream to be at a top region of the absorber.

21. The method of claim 20, wherein the solvent temperature is lowered using at least one of recycling and intercooling of the solvent solution and recycling of the solvent solution.

22. The solvent solution of claim 20, wherein the catalyst is a biocatalyst.

23. The solvent solution of claim 20, wherein the biocatalyst is carbonic anhydrase or an analog thereof.

24. The solvent solution of claim 20, wherein the amine solvent has a theoretical cyclic capacity greater than or equal to about 1 mole/liter.

25. The solvent solution of claim 20, wherein the amine solvent has an acid dissociation constant (pKa) greater than or equal to about 9 and less than or equal to about 10.5.

26. The solvent solution of claim 20, wherein the amine solvent is selected from the group including DMEA (dimethylethanolamine), DEEA (diethylethanolamine), and DMgly (dimethylglycine).
